(19) United States
(12) Patent Application Publication
(10) Pub. No.: US 2006/0040909 A1
(43) Pub. Date: Feb. 23, 2006

(54) Selenophene compounds

(75) Inventors: Shyh-Fong Chen, Taipei (TW); Wan-Ping Hsieh, Hsinchu City (TW); Pao-Chiung Hong, Taipei County (TW); Shan-Yen Chou, Taipei (TW); Shin-Hun Juang, Taipei (TW); Yu-Chen Li, Nantou (TW); Shieh-Shung T. Chen, Morganville, NJ (US); Leeyuan Huang, Watchung, NJ (US); Ching-Jer Chang, West Lafayette, IN (US); Pi-Chen Hsu, Taipei (TW); Cheng-Li Hsu, Taichung (TW)

Correspondence Address: FISH & RICHARDSON PC, P.O. BOX 1022, MINNEAPOLIS, MN, 55440-1022, US

(73) Assignee: DEVELOPMENT CENTER FOR BIOTECHNOLOGY

(21) Appl. No.: 11/208,695

(22) Filed: Aug. 22, 2005

Related U.S. Application Data
(60) Provisional application No. 60/603,773, filed on Aug. 23, 2004.

(51) Int. Cl. A61K 31/4025; A61K 31/381; A61K 31/343; A61K 31/33

(52) U.S. Cl. 514/183; 514/422; 514/444

(57) ABSTRACT

This invention relates to a method of treating liver cancer, comprising administering to a subject in need thereof an effective amount of a compound of formula (I): 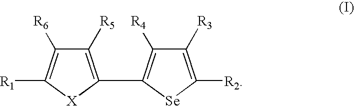
In formula (I), each of R₁ and R₂, independently, is H, C₁-C₁₀ alkyl, C₃-C₂₀ cycloalkyl, C₃-C₂₀ heterocycloalkyl, aryl, heteroaryl, or C(O)Rₐ; X is Se, S, O, or NR_{b}; and each of R₃, R₄, R₅, and R₆, independently, is H, C₁-C₁₀ alkyl, C₃-C₂₀ cycloalkyl, C₃-C₂₀ heterocycloalkyl, aryl, or heteroaryl; in which each of Rₐ and R_{b}, independently, is H or C₁-C₁₀ alkyl.

### CROSS REFERENCE TO RELATED APPLICATION

Pursuant to 35 U.S.C. § 119(e), this application claims priority to U.S. Provisional Application Ser. No. 60/603,773, filed Aug. 23, 2004, the contents of which are hereby incorporated by reference.

### BACKGROUND

Selenophene compounds are selenium-containing heterocyclic compounds that are structurally analogous to naturally occurring thiophene, furan, and pyrrole compounds. They have been investigated extensively for their efficacy in treating cancers.

### SUMMARY

This invention is based on the unexpected discovery that certain selenophene compounds are particularly effective in treating liver cancer.

In one aspect, this invention features a method of treating liver cancer. The method includes administering to a subject in need thereof an effective amount of a compound of formula (I): 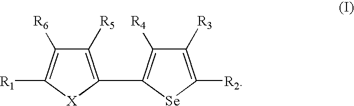
In the above formula, each of R₁ and R₂, independently, is H, C₁-C₁₀ alkyl, C₃-C₂₀ cycloalkyl, C₃-C₂₀ heterocycloalkyl, aryl, heteroaryl, or C(O)Rₐ; X is Se, S, O, or NR_{b}; and each of R₃, R₄, R₅, and R₆, independently, is H, C₁-C₁₀ alkyl, C₃-C₂₀ cycloalkyl, C₃-C₂₀ heterocycloalkyl, aryl, or heteroaryl; in which each of Rₐ and R_{b}, independently, is H or C₁-C₁₀ alkyl.

For example, one can administer to a subject having liver cancer a compound of
formula (I), in which one of R₁ and R₂ is 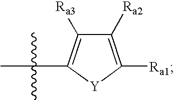
Y is Se, S, O, or NR_{b1}; and each of Rₐ₁, Rₐ₂, and Rₐ₃, independently, is H, C₁-C₁₀ alkyl, C₃-C₂₀ cycloalkyl, C₃-C₂₀ heterocycloalkyl, aryl, heteroaryl, or C(O)R; each of R_{b1} and R, independently, being H or C₁-C₁₀ alkyl. In this compound, the other of R₁ and R₂ can be H, CH₂OH, or CHO; each of R₃, R₄, R₅, and R₆, independently, can be H or CH₂OH; and each of Rₐ₁, Rₐ₂, and Rₐ₃, independently, can be H, CH₂OH, CHO, or

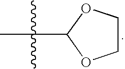

As another example, one can administer to a subject having liver cancer a compound of formula (I), in which each of R₁ and R₂, independently, is H or CH₂OH and R₃, R₄, R₅, and R₆, independent, is H or CH₂OH.

Shown below are the structures of compounds 1-23, exemplary compounds of formula (I):

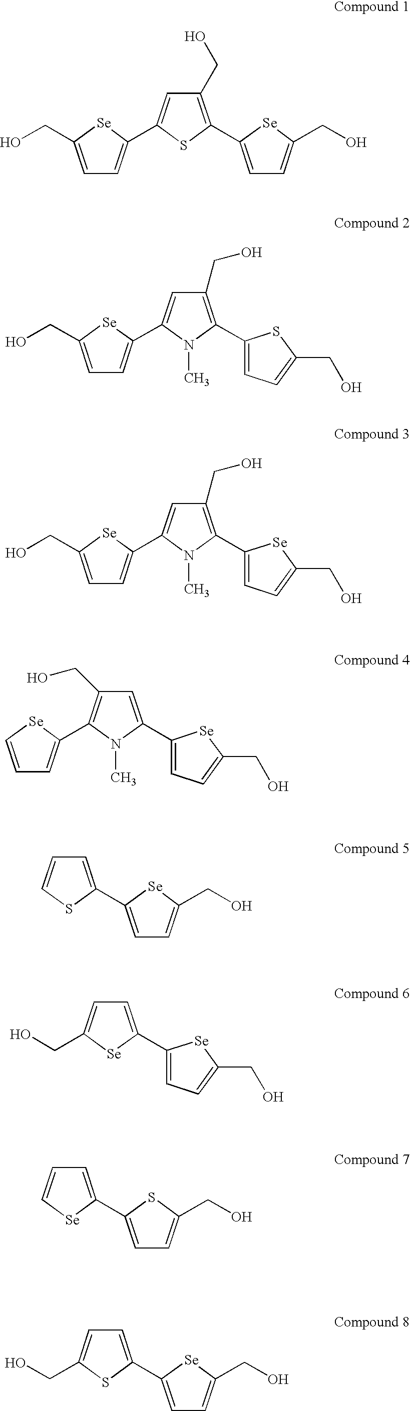

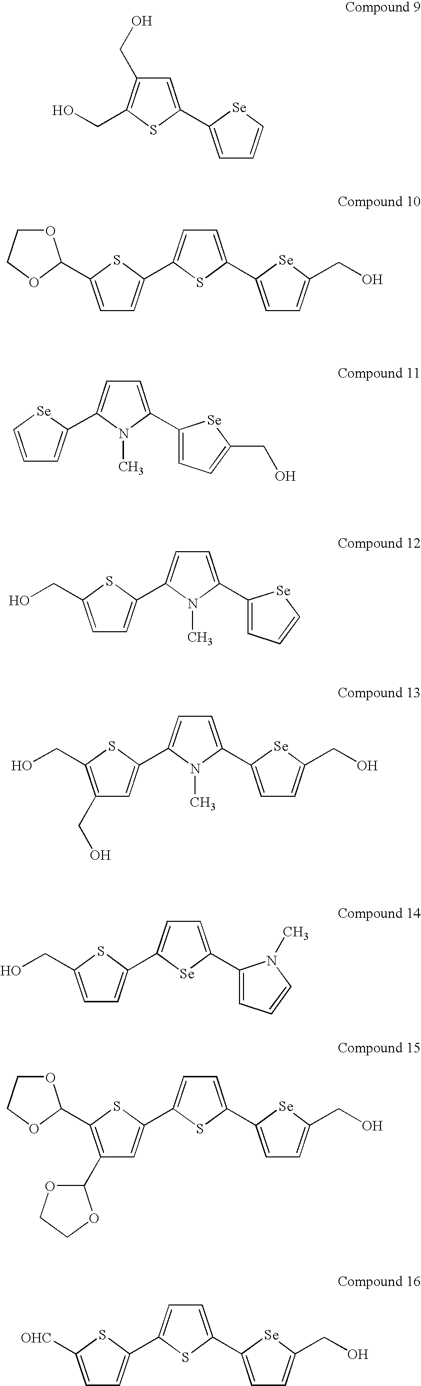

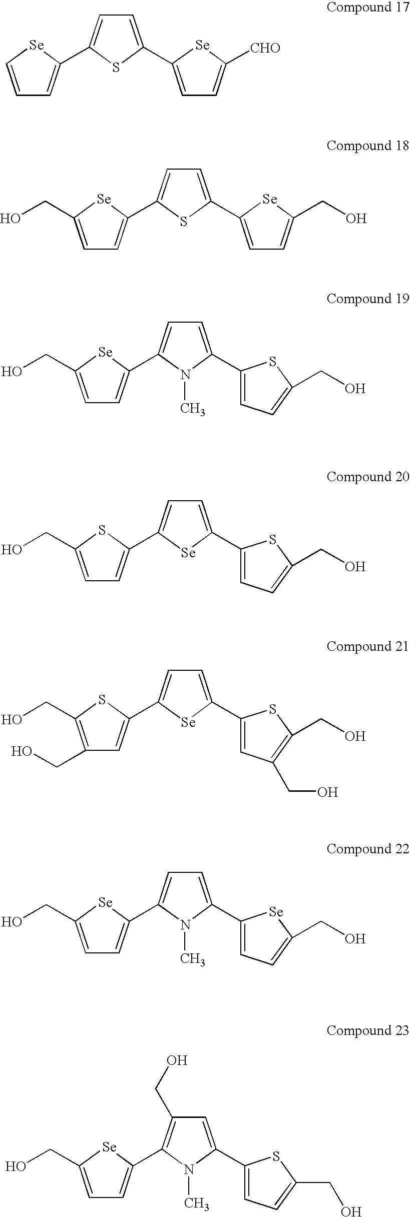

The term “treating” mentioned herein refers to administering one or more selenophene compounds described above to a subject, who has liver cancer, a symptom of liver cancer, or a predisposition toward liver cancer, with the purpose to confer a therapeutic effect, e.g., to cure, relieve, alter, affect, ameliorate, or prevent liver cancer, the symptom of it, or the predisposition toward it. The term “an effective amount” refers to the amount of one or more active selenophene compounds described above that is required to confer a therapeutic effect on a treated subject.

The term “alkyl” refers to a saturated or unsaturated, linear or branched, non-aromatic hydrocarbon moiety, such as —CH₃, —CH₂—, —CH₂—CH═CH—, or branched —C₃H₇. The term “cycloalkyl” refers to a saturated or unsaturated, non-aromatic, cyclic hydrocarbon moiety, such as cyclohexyl or cyclohexen-3-yl. The term “heterocycloalkyl” refers to a saturated or unsaturated, non-aromatic, cyclic moiety having at least one ring heteroatom (e.g., N, O, or S), such as 4-tetrahydropyranyl or 4-pyranyl. The term “aryl” refers to a hydrocarbon moiety having one or more aromatic rings. Examples of an aryl moiety include phenyl, phenylene, naphthyl, naphthylene, pyrenyl, anthryl, and phenanthryl. The term “heteroaryl” refers to a moiety having one or more aromatic rings that contain at least one heteroatom (e.g., N, O, or S). Examples of a heteroaryl moiety include furyl, furylene, fluorenyl, pyrrolyl, thienyl, oxazolyl, imidazolyl, thiazolyl, pyridyl, pyrimidinyl, quinazolinyl, quinolyl, isoquinolyl and indolyl.

Alkyl, cycloalkyl, heterocycloalkyl, aryl, and heteroaryl mentioned herein include both substituted and unsubstituted moieties, unless specified otherwise. Possible substituents on cycloalkyl, heterocycloalkyl, aryl, and heteroaryl include C₁-C₁₀ alkyl, C₂-C₁₀ alkenyl, C₂-C₁₀ alkynyl, C₃-C₈ cycloalkyl, C₅-C₈ cycloalkenyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, hydroxyl, halogen, thio, C₁-C₁₀ alkylthio, arylthio, C₁-C₁₀ alkylsulfonyl, arylsulfonyl, acylamino, aminoacyl, amidino, guanidine, ureido, cyano, nitro, acyl, acyloxy, carboxyl, and carboxylic ester. On the other hand, possible substituents on alkyl include all of the above-recited substituents except C₁-C₁₀ alkyl, C₂-C₁₀ alkenyl, and C₂-C₁₀ alkynyl. Cycloalkyl, heterocycloalkyl, aryl, and heteroaryl can also be fused with each other.

In another aspect, this invention features a compound selected from the group consisting of compounds 1-10, 12-16, 19, 21, and 23 described above.

In addition, this invention encompasses a pharmaceutical composition that contains an effective amount of at least one of the above-mentioned selenophene compounds and a pharmaceutically acceptable carrier.

The selenophene compounds described above include the compounds themselves, as well as their salts, prodrugs, and solvates, if applicable. A salt, for example, can be formed between an anion and a positively charged group (e.g., amino) on a selenophene compound. Examples of suitable anions include chloride, bromide, iodide, sulfate, nitrate, phosphate, citrate, methanesulfonate, trifluoroacetate, and acetate. Likewise, a salt can also be formed between a cation and a negatively charged group (e.g., carboxylate) on a selenophene compound. Examples of suitable cations include sodium ion, potassium ion, magnesium ion, calcium ion, and an ammonium cation such as tetramethylammonium ion. The selenophene compounds also include those salts containing quaternary nitrogen atoms. Examples of prodrugs include esters and other pharmaceutically acceptable derivatives, which, upon administration to a subject, are capable of providing active selenophene compounds described above. A solvate refers to a complex formed between an active selenophene compound described above and a pharmaceutically acceptable solvent. Examples of pharmaceutically acceptable solvents include water, ethanol, isopropanol, ethyl acetate, acetic acid, and ethanolamine.

Also within the scope of this invention is a composition containing one or more of the selenophene compounds described above for use in treating liver cancer, and the use of such a composition for the manufacture of a medicament for the just-mentioned treatment.

The details of one or more embodiments of the invention are set forth in the description below. Other features, objects, and advantages of the invention will be apparent from the description and from the claims.

### DETAILED DESCRIPTION

Selenophenes are selenium-containing heterocyclic compounds that are analogs of naturally occurring thiophene, furan, and pyrrole compounds.

The selenophene compounds described above can be prepared by methods well known in the art, as well as the synthetic routes disclosed herein. For example, Schemes 1-4 below depict typical synthetic routes for synthesizing exemplary compounds 1-23. Details of preparation of these compounds are provided in Examples 1-23, respectively.

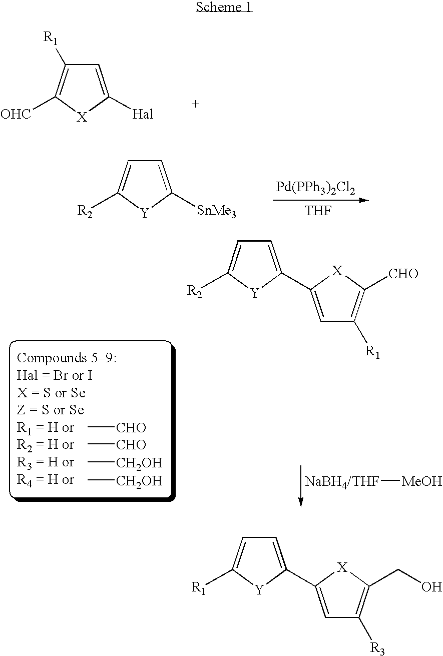

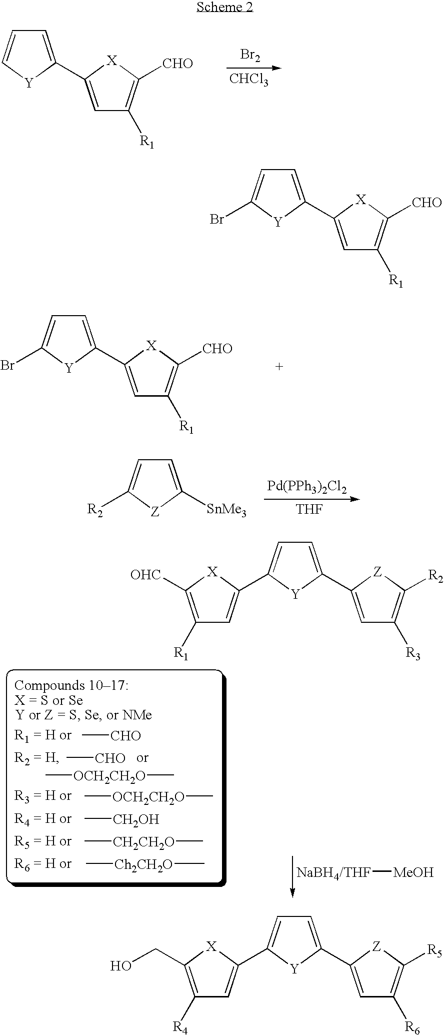

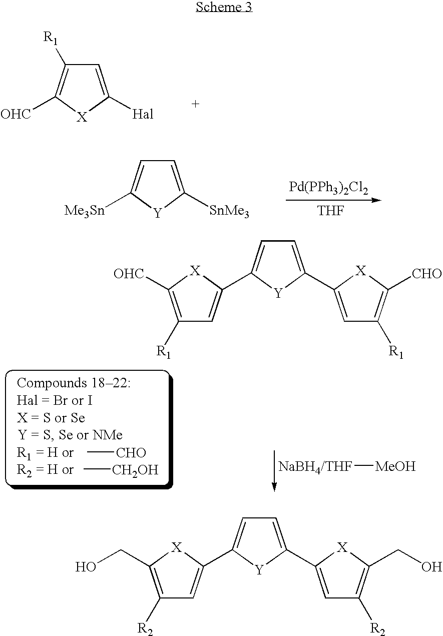

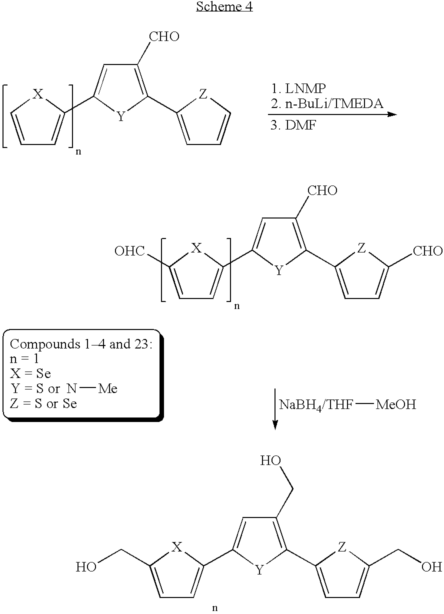

Referring to Scheme 1, one can obtain a selenophene compound containing two five-membered rings (e.g., thiophene, pyrrole, and selenophene rings) by reacting a compound containing a first five-membered ring substituted with a halide group with a compound containing a second five-membered ring substituted with a trimethylstannyl group. Referring to Scheme 2, the selenophene compound obtained above can be further halogenated and then bonded to a compound containing a third five-membered ring, thereby forming a selenophene compound containing three five-membered rings via the just-mentioned coupling reaction. Referring to Scheme 3, a selenophene compound containing three five-membered rings can also be obtained by coupling one equivalent of a compound containing a bis-trimethylstannyl substituted five-membered ring with two equivalents of a compound containing a halogen substituted five-membered ring. Referring to Scheme 4, a selenophene compound containing three five-membered rings can be further modified to introduce aldehyde or hydroxymethyl groups.

A selenophene compound thus synthesized can be further purified by a known method such as column chromatography, high-pressure liquid chromatography, or recrystallization.

Other selenophene compounds described in the Summary section above can be prepared using other suitable starting materials following the synthetic routes disclosed herein and other synthetic methods known in the art. The methods described above may also additionally include steps, either before or after the steps described specifically herein, to add or remove suitable protecting groups in order to ultimately allow synthesis of the selenophene compounds described above. In addition, various synthetic steps may be performed in an alternate sequence or order to give the desired selenophene compounds. Synthetic chemistry transformations and protecting group methodologies (protection and deprotection) useful in synthesizing applicable selenophene compounds described above are known in the art and include, for example, those described in R. Larock, Comprehensive Organic Transformations, VCH Publishers (1989); T. W. Greene and P. G. M. Wuts, Protective Groups in Organic Synthesis, 2nd Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The selenophene compounds mentioned herein may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

Also within the scope of this invention is a pharmaceutical composition contains an effective amount of at least one selenophene compound described above and a pharmaceutical acceptable carrier. Further, this invention covers a method of administering an effective amount of one or more of the selenophene compounds described above to a patient having liver cancer. Effective doses will vary, as recognized by those skilled in the art, depending on the types of diseases treated, route of administration, excipient usage, and the possibility of co-usage with other therapeutic treatment.

To practice the method of the present invention, a composition having one or more selenophene compounds described above can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having one or more selenophene compounds described above can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of a compound of the invention. Examples of other carriers include colloidal silicon oxide, magnesium stearate, cellulose, sodium lauryl sulfate, and D&C Yellow # 10.

The selenophene compounds described above can be preliminarily screened for their efficacy in treating liver cancer by an in vitro assay (see Example 24 below) and then confirmed by an in vivo assay (see Example 25 below). Other methods will also be apparent to those of ordinary skill in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1:

### Preparation of Compound 1: 2,5-bis-((5′-hydroxymethyl)-2′-selenyl)-3-hydroxylthiophene

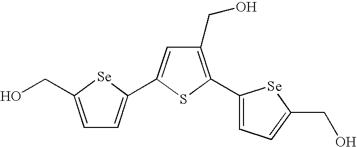

To a solution of N-methylpiperazine (0.08 ml, 0.72 mmol) in 1 ml THF at 0° C. was added n-butyllithium (0.4 ml of a 1.6 M solution in hexane, 0.64 mmol). After stirring for 15 min, a solution of 2,5-di-selenophen-2-yl-thiophene-3-carbaldehyde (0.23 g, 0.64 mmol) in 7 ml THF was added to the above solution and the mixture was stirred at 0° C. for another 15 min. A solution of TMEDA (0.6 ml, 4.0 mmol) and n-BuLi (2.5 ml, 4.0 mmol) in hexane was then added to the above mixture. After stirring at 0° C. for 2 hours, the reaction mixture was cooled to −78° C. and DMF (0.8 ml, 10 mmol) was added. The mixture was allowed to come to room temperature during a 2-hour period. It was then poured onto cold water and extracted with ethyl acetate. The organic extracts were combined, washed with brine, dried, and concentrated. The crude product thus obtained was purified by flash column chromatography (silica gel, EtOAc-hexane, 1:3) to give 81 mg of a trialdehyde (yield: 30%).

To a solution of the trialdehyde (24 mg, 0.05 mmol) in 20 ml THF/MeOH (1:1), was added an excessive amount of NaBH₄ at 0° C. The mixture was stirred for 1 hour and then diluted with ethyl acetate. The organic layer was separated, washed with brine, dried, and concentrated. The crude product thus obtained was purified by flash column chromatography (silica gel, EtOAc-hexane, 1:3) to give 18 mg of the compound 1 (yield: 30%).

¹H NMR (500 MHz, Acetone-d₆): δ 7.28(d, 1H), 7.25(d, 1H), 7.23(s, 1H), 7.13(d, 1H), 7.06(d, 1H), 4.83(m, 4H), 4.67(d, 2H), 4.65(d, 2H), 4.32(d, 1H).

LC-MS found: 419 (40%), 417 (M⁺+1-18, 100%), 415 (98%), 413 (50%), 411 (20%).

### EXAMPLE 2

### Preparation of Compound 2: 2-((5′-hydroxymethyl)-2′-thienyl)-5-((5″-hydroxymethyl)-2″-selenyl)-3-hydroxylmethyl-N-methyl-pyrrole

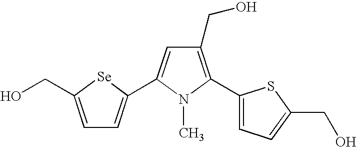

Compound 2 was prepared in a manner similar to that described in Example 1 using a suitable monoformyl-substituted triheteroaryl compound as a starting material.

¹H NMR (500 MHz, Acetone-d₆): δ 7.10(m, 2H), 7.00(m, 2H), 6.36(s, 1H), 4.80(m, 4H), 4.42(m, 2H), 3.65(s, 3H).

### EXAMPLE 3

### Preparation of Compound 3: 2,5-bis-(5′-hydroxymethyl)-2′-selenyl)-3-hydroxyl-N-methyl-pyrrole

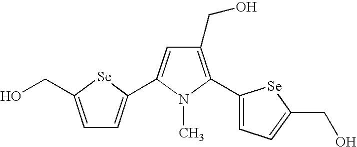

Compound 3 was prepared in a manner similar to that described in Example 1 using a suitable monoformyl-substituted triheteroaryl compound as a starting material.

¹H NMR (500 MHz, Acetone-d₆): δ 7.12(m, 4H), 6.36(s, 1H), 4.82(m, 4H), 4.43(d, 2H), 3.67(s, 3H).

### EXAMPLE 4

### Preparation of Compound 4: 2-(2′-thienyl)-5-((5″-hydroxymethyl)-2″-selenyl)-3-hydroxymethyl-N-methyl-pyrrole

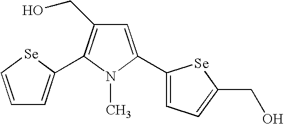

Compound 4 was prepared in a manner similar to that described in Example 1 using a suitable monoformyl-substituted triheteroaryl compound as a starting material.

¹H NMR (500 MHz, Acetone-d₆): δ 8.26(q, 1H), 7.38(q, 1H), 7.31(q, 1H), 7.11(m, 2H), 6.36(s, 1H), 4.80(s, 2H), 4.42(s, 2H), 3.66(s, 3H).

### EXAMPLE 5

### Preparation of Compound 5: 2-(5′-hydroxymethyl-2′-selenyl)-thiophene

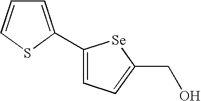

To a solution of 5-iodo-selenophene-2-carbaldehyde (0.8 g, 2.8 mmol) and trimethyl-thiophen-2-yl-stannane (0.83 g, 3.4 mmol) in THF (10 ml) was added dichlorobis(triphenylphosphine)palladium (0.09 g, 0.13 mmol). The mixture was heated at 65° C. for 16 hours and then concentrated. The residue thus obtained was purified by flash column chromatography (silica gel, EtOAc-hexane, 1:10) to give a mono-substituted aldehyde (0.51 g, yield: 75%).

To a solution of the mono-substituted aldehyde obtained above (150 mg, 0.62 mmol) in 10 ml THF/MeOH (1:1) was added an excessive amount of NaBH₄ at 0° C. After the solution was stirred for 1 hour, ethyl acetate was added. The organic layer was then separated, washed with brine, dried, and concentrated. The residue thus obtained was purified by recrystallization from ethyl acetate/hexane to give compound 5 (130 mg, yield: 85%).

¹H NMR (500 MHz, Acetone-d₆): δ 7.36(d, 1H), 7.22(m, 1H), 7.16(d, 1H), 7.03(m, 2H), 4.78(d, 2H), 4.58(t, 1H).

LC-MS found: 229(30%), 227(M⁺+1-18, 100%), 225(80%)

### EXAMPLE 6

### Preparation of Compound 6: 2-(5′-hydroxymethyl-2′-selenyl)-5-hydroxymethyl-thiophene

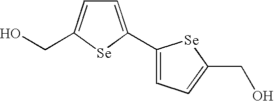

Compound 6 was prepared in a manner similar to that described in Example 5.

¹H NMR (500 MHz, Acetone-d₆): δ 7.12(d, 2H), 7.02(d, 2H), 4.78(s, 4H).

### EXAMPLE 7

### Preparation of Compound 7: 2-(5′-hydroxymethyl-2′-thienyl)-selenophene

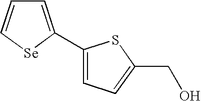

Compound 7 was prepared in a manner similar to that described in Example 5.

¹H NMR (500 MHz, Acetone-d₆): δ 8.03(d, 1H), 7.35(d, 1H), 7.28(m, 1H), 7.07(d, 1H), 6.90 (d, 1H), 4.76(d, 2H), 4.47(t, 1H).

LC-MS found: 229(30%), 227(M⁺+1-18, 100%), 225(70%).

### EXAMPLE 8

### Preparation of Compound 8: 2-(5′-hydroxymethyl-2′-selenyl)-5-hydroxymethyl-thiophene

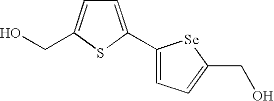

Compound 8 was prepared in a manner similar to that described in Example 5.

¹H NMR (500 MHz, Acetone-d₆): δ 7.18(d, 1H), 7.04(d, 1H), 7.01(d, 1H), 6.89(d, 1H), 4.80 (d, 2H), 4.75(d, 2H), 4.58(t, 1H), 4.44(t, 1H).

LC-MS found: 259(20%), 257(M⁺+1-18, 100%), 255(60%).

### EXAMPLE 9

### Preparation of Compound 9: 5-(2′-selenyl)-2,5-di(hydroxymethyl)-thiophene

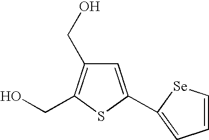

Compound 9 was prepared in a manner similar to that described in Example 5.

¹H NMR (500 MHz, Acetone-d₆): δ 8.02(q, 1H), 7.34(t, 1H), 7.27(t, 1H), 7.13(s, 1H), 4.77(d, 2H), 4.59(d, 2H), 4.42(t, 1H), 4.10(t, 1H).

LC-MS found: 259(20%), 257(M⁺+1-18,100%), 255(70%).

### EXAMPLE 10

### Preparation of Compound 10: 2-((5′-hydroxymethyl)-2′-selenyl)-5-(5″-ethyleneketal)-2″-thienyl)-thiophene

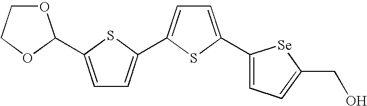

To a solution of 5-thiophen-2-yl-selenophene-2-carbaldehyde (0.6 g, 2.49 mmol) in chloroform (20 ml) was added sodium bicarbonate (0.252 g, 3.0 mmol), followed by addition of a solution of bromine (0.437 g, 2.74 mmol) in chloroform (20 ml) in a dropwise manner over a period of 30 minutes. The reaction mixture was refluxed for 4 hours, cooled down to room temperature, and filtered. The filtrate was washed with brine and dried over MgSO₄. The solvent was removed under vacuum and the residue thus obtained was purified by flash column chromatography (silica gel, EtOAc-hexane, 1:15) to give intermediate 1,5-(5-bromo-thiophen-2-yl)-selenophene-2-carbaldehyde (0.60 g, yield: 76%).

To a solution of intermediate 1 (0.23 g, 0.72 mmol) and (5-[1,3]dioxolan-2-yl-thiophen-2-yl)-trimethylstannane (0.275 g, 0.86 mmol) in THF (10 ml) was added dichlorobis(triphenylphosphine)palladium (0.025 g, 0.04 mmol). The mixture was refluxed at 75° C. for 16 hours and then concentrated. The residue thus obtained was purified by flash column chromatography (silica gel, EtOAc-hexane, 1:5) to give intermediate 2 (0.165 g, yield: 58%).

To a solution of intermediate 2 (80 mg, 0.02 mmol) in 20 ml THF/MeOH (1:1) was added an excessive amount of NaBH₄ at 0° C. The solution was stirred for 1 hour and then diluted with ethyl acetate. The organic layer was separated, washed with brine, dried over MgSO₄, and concentrated. The residue thus obtained was purified by recrystallization from ethyl acetate/hexane to give compound 10 (70 mg, yield: 87%).

¹H NMR (500 MHz, Acetone-d₆): δ 7.28(d, 1H), 7.21(d, 1H), 7.19(d, 1H), 7.15(m, 2H), 7.07(d, 1H), 6.06(s, 1H), 4.82(d, 2H), 4.66(t, 1H), 4.12(m, 2H), 4.02(m, 2H).

LC-MS found: 401(30%), 399(M⁺+1, 100%), 397(60%).

### EXAMPLE 11

### Preparation of Compound 11: 2-(2′-thienyl-2′-selenyl)-5-(5″-hydroxymethy-2″-selenyl)-N-methyl-pyrrole

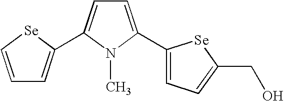

Compound 11 was prepared in a manner similar to that described in Example 10.

¹H NMR (500 MHz, Acetone-d₆): δ 8.10(d, 1H), 7.34(d, 1H), 7.29(d, 1H), 7.12(m, 2H), 6.28(d, 1H), 6.25(d, 1H), 4.80(d, 2H), 3.77(s, 3H).

LC-MS found: 372(M⁺+1, 100%), 370(50%),368(15%).

### EXAMPLE 12

### Preparation of Compound 12: 2-(5′-hydroxymethyl-2′-thienyl)-5-( 2″-selenyl)-N-methyl-pyrrole

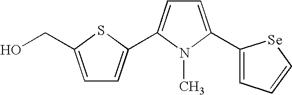

Compound 12 was prepared in a manner similar to that described in Example 10.

¹H NMR (500 MHz, Acetone-d₆): δ 7.94(dd, 1H), 7.20(dd, 1H), 7.15(dd, 2H), 6.85(d, 1H), 6.83 (d, 1H), 6.15(d, 1H), 6.13(d, 1H), 4.64(d, 2H), 4.32(t, 1H), 3.63(s, 3H).

LC-MS found: 324(M⁺+1, 100%), 322(60%),320(20%).

### EXAMPLE 13

### Preparation of Compound 13: 2-(4′,5′-dihydroxymethyl-2′-thienyl)-5-(5″-hydroxymethyl-2″-selenyl)-N-methyl-pyrrole

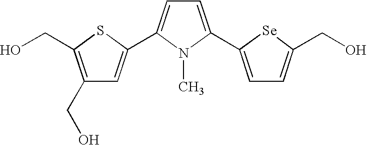

Compound 13 was prepared in a manner similar to that described in Example 10.

¹H NMR(500 MHz, Acetone-d₆): δ 7.12(d, 2H), 7.08(s, 1H), 6.27(s, 2H), 4.82(d, 2H), 4.80(d, 2H), 4.63(d, 2H), 4.46(t, 1H), 4.11(t, 1H), 3.79(s, 3H), 3.42(t, 1H)

LC-MS found: 384(M⁺+1, 30%), 368(30%), 366(M⁺+1-H₂O, 100%), 364(70%).

### EXAMPLE 14

### Preparation of Compound 14: 2-(5′-dihydroxymethyl-2′-thienyl)-5-(2″-N-methylpyrrolyl)-selenophene

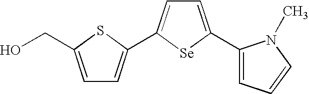

Compound 14 was prepared in a manner similar to that described in Example 10.

¹H NMR (500 MHz, Acetone-d₆): δ 7.31(d, 1H), 7.16(d, 1H), 7.05(d, 2H), 6.91(dd, 1H), 6.82 (t, 1H), 6.29(dd, 1H), 6.06(dd, 1H), 4.74(s, 2H), 3.79(s, 3H).

### EXAMPLE 15

### Preparation of Compound 15: 2-(4′,5′-diethyleneketal-2′-thienyl)-5-(5″-hydroxymethyl-2″ selenyl)-thiophene

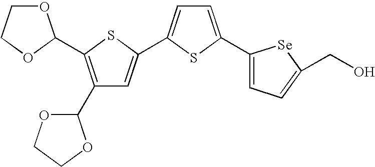

Compound 15 was prepared in a manner similar to that described in Example 10.

¹H NMR (500 MHz, Acetone-d₆): δ 7.28(d, 1H), 7.23(s, 1H), 7.22(d, 1H), 7.14(d, 1H), 7.07(d, 1H), 6.30(s, 1H), 5.98(s, 1H), 4.82(d, 2H), 4.65(t, 1H), 4.13(m, 4H), 4.01(m, 4H).

LC-MS found: 473(30%), 371(M⁺+1, 100%), 469(80%)

### EXAMPLE 16

### Preparation of Compound 16: 2-(5′-formyl -2′-thienyl)-5-(5″-hydroxymethyl-2″-selenyl)-thiophene

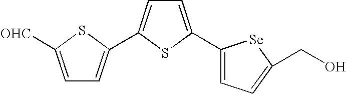

Compound 16 was prepared in a manner similar to that described in Example 10.

¹H NMR (500 MHz, Acetone-d₆): δ9.92(s, 1H), 7.92(d, 1H), 7.47(m, 2H), 7.34(d, 1H), 7.21(d, 1H), 7.08(d, 1H), 4.81(d, 2H), 4.69(t, 1H).

LC-MS found: 357(30%), 355(M⁺+1, 100%), 353(80%).

### EXAMPLE 17

### Preparation of Compound 17: 2-(2′-thienyl)-5-(5″-formyl-2″-selenyl)-thiophene

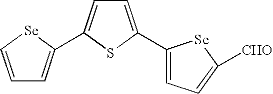

Compound 17 was prepared in a manner similar to that described in Example 10.

¹H NMR (500 MHz, Acetone-d₆): δ 9.80(s, 1H), 8.14(m, 2H), 7.61(d, 1H), 7.51(t, 1H), 7.45(d, 1H), 7.32(q, 1H), 7.26(d, 1H).

LC-MS found: 375(20%), 373(M⁺+1, 100%), 371(70%),369(35%).

### EXAMPLE 18

### Preparation of Compound 18: 2,5-bis-((5′-hydroxymethyl)-2′-selenyl)-thiophene

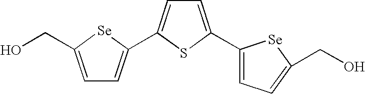

To a solution of 5-iodo-selenophene-2-carbaldehyde (0.5 g, 1.22 mmol) and 2,5-bis-trimethylstannanyl-thiophene (0.76 g, 2.66 mmol) in THF (10 ml) was added dichlorobis(triphenylphosphine)palladium (45 mg, 0.06 mmol). The mixture was refluxed at 60° C. for 16 hours. The solvent was then removed under vacuum. The residue thus obtained was purified by flash column chromatography (silica gel, EtOAc-hexane, 1:3) to give a dialdehyde (0.584 g, yield: 55%).

To a solution of the dialdehyde obtained above (50 mg, 0.12 mmol) in 45 ml THF/MeOH (2:1) was added an excessive amount of NaBH₄ at 0° C. After the mixture was stirred for 1 hour, ethyl acetate was added. The organic layer was then separated, washed with brine, dried over MgSO₄, and concentrated. The residue thus obtained was purified by recrystallization from ethyl acetate/hexane to give compound 18 (41 mg, yield: 80 %).

¹H NMR (500 MHz, DMSO-d₆): δ 7.27(d, 2H), 7.14(s, 2H), 7.04(d, 2H), 5.66(t, 2H), 4.64(d, 4H).

LC-MS found: 389(50%), 387(M⁺+1-18, 100%), 385(90%),383(80 %), 381(30%).

### EXAMPLE 19

### Preparation of Compound 19: 2,5-bis-((5′-hydroxymethyl)-2′-selenyl)-N-methyl-pyrrole

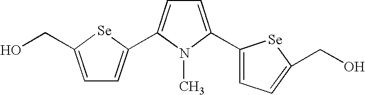

Compound 19 was prepared in a manner similar to that described in Example 18.

¹H NMR (500 MHz, Acetone-d₆): δ 7.13(m, 2H), 7.00(m, 2H), 6.27(s, 2H), 4.82(d, 2H), 4.79(d, 2H), 3.78(s, 3H).

LC-MS found: 356(10%), 354(M⁺+1, 50%), 352(30%), 350(10%), 336(M⁺+1-18, 100%).

### EXAMPLE 20

### Preparation of Compound 20: 2,5-bis-((5′-hydroxymethyl)-2′-thienyl)-selenophene

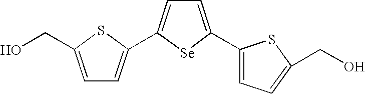

Compound 20 was prepared in a manner similar to that described in Example 18.

¹H NMR (500 MHz, Acetone-d₆): δ 7.14(s, 2H), 6.93(d, 2H), 6.77(d, 2H), 4.62(d, 4H), 4.36(t, 2H).

### EXAMPLE 21

### Preparation of Compound 21: 2,5-bis-((4′,5′-dihydroxymethyl)-2′-thienyl)-selenophene

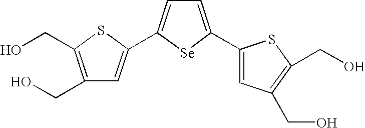

Compound 21 was prepared in a manner similar to that described in Example 18.

¹H NMR (500 MHz, Acetone-d₆): δ7.27(s, 2H), 7.14(s, 2H), 4.78(d, 4H), 4.59(d, 4H), 4.54(t, 2H), 4.20(t, 2H).

LC-MS found: 356(10%), 354(M⁺+1, 50%), 352(30%), 350(10%), 336(M⁺+1-18, 100%).

### EXAMPLE 22

### Preparation of Compound 22: 2,5-bis-((5′-dihydroxymethyl)-2′-selenyl)-N-methyl-pyrrole

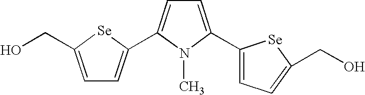

Compound 22 was prepared in a manner similar to that described in Example 18.

¹H NMR (500 MHz, Acetone-d₆): δ 7.10(m, 4H), 6.24(s, 2H), 4.80(d, 4H), 3.77(s, 3H).

### EXAMPLE 23

### Preparation of Compound 23: 2-((5′-hydroxymethyl)-2′-thienyl)-5(-(5″-hydroxymethyl)-2″-selenyl-)-4-hydroxymethyl-N-methyl-pyrrole

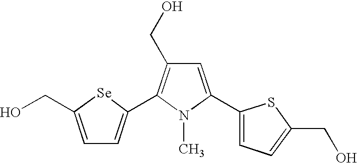

Compound 23 was prepared in a manner similar to that described in Example 1 using a suitable monoformyl-substituted triheteroaryl compound as a starting material.

¹H NMR (500 MHz, Acetone-d₆): δ 7.10(m, 2H), 7.00(m, 2H), 6.35(s, 1H), 4.80(m, 4H), 4.42(m, 2H), 3.64(s, 3H).

### EXAMPLE 24

### In vitro Cytotoxicity Assay

22 compositions (i.e., compounds 1, 3-22, and a mixture of compounds 2 and 23) were tested for their efficacy in inhibiting eight different tumor cells (i.e., Hep-3B liver cancer cells, MKN-45 gastric cancer cells, HCT-116 colon cancer cells, NPC-TW01 nasopharyngeal cancer cells, NCI-H226 lung cancer cells, A-498 kidney cancer cells, LNCap prostate cancer cells, and MCF-7 breast cancer cells). Typically, tumor cells were seeded at a density ranging from 2×10³ to 8×10³ cells/well in a 96-well plate 16 hours prior to treatment with a test composition. After incubation with the test composition at different concentrations for 72 hours, cells were incubated with a medium containing 0.2 mg/ml i.e., 3-(4,5-dimethythiazol-2-yl)-5-(3-carboxymethosyphenyl)-2-(4-sulfophenyl)-2H-tetrazolium (MTS) for 2 hours. The conversion of MTS to formazan by metabolically viable cells was measured by the absorbance at 490 nm in a 96-well microtiter plate reader. A control group containing untreated cells was used to determine the concentration of the test composition at which 50% of cell growth was inhibited (IC₅₀).

Unexpectedly, 12 compositions showed IC₅₀ values between 0.1 μM to 1 μM and 10 composition showed IC₅₀values between 0.01 μM and 0.1 μM in inhibiting Hep-3B liver cancer cells. They were 2 folds to 30 folds more potent in inhibiting Hep-3B liver cancer cells than in inhibiting HCT-116 colon cancer cells. 21 of these compositions were 4 folds to 500 folds more potent in inhibiting Hep-3B liver cancer cells than in inhibiting MKN-45 gastric cancer cells

### EXAMPLE 25

### In vivo Assay

Compound 3 was tested for its efficacy against Hep-3B liver cancer cells implanted in mice of two treated groups at a daily dosage of 50 mg/kg and 75 mg/kg. In a control group, mice bearing Hep-3B liver cancer cells were not treated with any test compound. Male athymic mice (Ncr nu/nu) of 4-6 weeks of age and weighing 20-25 g were used. Mice were housed in cages in ventilated cabinets and provided sterilized pellet diet and sterile water ad libitum. The temperature was kept between 23-25° C. and a humidity of about 50±10%. Light of the housing area was on a 12-hour light/dark cycle. All experimentation was performed under the IACUC guidelines adopted by the Development Center for Biotechnology, Taiwan.

Hep-3B cells were transplanted subcutaneously into athymic mice. In the treated groups, when the tumor reached a volume of about 50 mm³, mice were treated with compound 3 at a dosage of 50 mg/kg or 75 mg/kg once every two days for three times. The treated groups and the control group each had 7 mice. Compound 3 was administered intraperitoneally. The sizes of tumors were measured using a caliper at least twice a week and the tumor volume (TV) was calculated. Body weights were also monitored along with tumor volume measurement. The percentage of tumor growth was determined by dividing the mean TV of the treated group by the mean TV of a control group.

The results showed that, after three doses of 50 mg/kg, mean TV in the mice reduced 37% at the 14^{th} day. After three doses of 75 mg/kg, mean TV reduced 63% at the 14^{th} days.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method of treating liver cancer, comprising administering to a subject in need thereof an effective amount of a compound of formula (I): 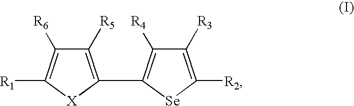
wherein
each of R₁ and R₂, independently, is H, C₁-C₁₀ alkyl, C₃-C₂₀ cycloalkyl, C₃-C₂₀ heterocycloalkyl, aryl, heteroaryl, or C(O)Rₐ;
X is Se, S, O, or NR_{b}; and
each of R₃, R₄, R₅, and R₆, independently, is H, C₁-C₁₀ alkyl, C₃-C₂₀ cycloalkyl, C₃-C₂₀ heterocycloalkyl, aryl, or heteroaryl;
in which each of Rₐ and R_{b}, independently, is H or C₁-C₁₀ alkyl.

2. The method of claim 1, wherein one of R₁ and R₂ is 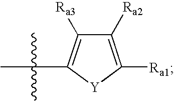
which Y is Se, S, O, or NR_{b1}; and each of Rₐ₁, Rₐ₂, and Rₐ₃, independently, is H, C₁-C₁₀ alkyl, C₃-C₂₀ cycloalkyl, C₃-C₂₀ heterocycloalkyl, aryl, heteroaryl, or C(O)R; each of R_{b1} and R, independently, being H or C₁-C₁₀ alkyl.

3. The method of claim 2, wherein Y is S.

4. The method of claim 3, wherein X is S or NCH₃.

5. The method of claim 4, wherein the other of R₁ and R₂ is H or CH₂OH.

6. The method of claim 5, wherein each of R₃, R₄, R₅, and R₆, independently, is H or CH₂OH.

7. The method of claim 6, wherein each of Rₐ₁, Rₐ₂, and Rₐ₃, independently, is H, CH₂OH, CHO, or

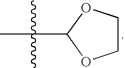

8. The method of claim 7, wherein the compound is one of compounds 2, 10, 12, 13, 15, 16, and 19-21.

9. The method of claim 2, wherein Y is Se.

10. The method of claim 9, wherein X is S or NCH₃.

11. The method of claim 10, wherein the other of R₁ and R₂ is H, CH₂OH, or CHO.

12. The method of claim 11, wherein each of R₃, R₄, R₅, and R₆, independently, is H or CH₂OH.

13. The method of claim 12, wherein each of Rₐ₁, Rₐ₂, and Rₐ₃, independently, is H or CH₂OH.

14. The method of claim 13, wherein the compound is one of compounds 1, 3, 4, 11, 17, 18, and 22.

15. The method of claim 2, wherein Y is NCH₃.

16. The method of claim 15, wherein X is S.

17. The method of claim 16, wherein the other of R₁ and R₂ is CH₂OH.

18. The method of claim 17, wherein each of R₃, R₄, R₅, and R₆ is H.

19. The method of claim 18, wherein each of Rₐ₁, Rₐ₂, and Rₐ₃ is H.

20. The method of claim 1, wherein each of R₁ and R₂, independently, is H or CH₂OH.

21. The method of claim 20, wherein X is S or Se.

22. The method of claim 21, wherein each of R₃, R₄, R₅, and R₆, independent, is H or CH₂OH.

23. The method of claim 22, wherein the compound is one of compounds 5-9.
